(19) United States
(12) Patent Application Publication
(10) Pub. No.: US 2012/0283233 A1
(43) Pub. Date: Nov. 8, 2012

(54) CARRIER COMPOSITION

(76) Inventors: Paul David Gavin, Chadstone (AU); Mahmoud El-Tamimy, Meadow Heights (AU); Roksan Libinaki, Chadstone (AU); Mohammad Reza Mozafari, Clayton (AU)

(21) Appl. No.: 13/501,498

(22) PCT Filed: Feb. 4, 2011
(86) PCT No.: PCT/AU11/00122

§ 371 Date: Apr. 12, 2012

Related U.S. Application Data
(60) Provisional application No. 61/306,115, filed on Feb. 19, 2010.

(30) Foreign Application Priority Data
Feb. 5, 2010 (AU) 2010900463
Jun. 4, 2010 (AU) 2010902463

(51) Int. Cl. A61K 47/24; A61K 31/167; A61K 31/573; A61K 31/407; A61K 31/57; A61K 47/26; A61K 31/196

(52) U.S. Cl. 514/180; 514/788; 514/772; 514/785; 514/777; 514/786; 514/626; 514/567; 514/412; 514/179

(57) ABSTRACT

The present invention relates to a carrier composition comprising a phosphate compound of an electron transfer agent and a polar aprotic solvent. Biologically active compounds formulated with the carrier composition have been shown to have improved properties.

### TECHNICAL FIELD

The present invention relates to carrier compositions for delivery of biologically active compounds.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

Drug delivery is the method or process of administering a pharmaceutical compound to achieve a therapeutic effect in humans and animals.

Drug delivery technologies have been developed to improve bioavailability, safety, duration, onset or release, of the pharmaceutical compound.

When developing drug delivery technologies, problems likely to be encountered include compatibility of the drug delivery system and the pharmaceutical compound, maintaining an adequate and effective duration, potential for side effects, and meeting patient convenience and compliance. As a consequence, many drug delivery technologies fall short of desired improvements and requirements.

Accordingly, there is still a need for alternate drug delivery systems that effectively deliver drugs.

### SUMMARY

It has surprisingly been found that a carrier composition comprising a phosphate compound of an electron transfer agent and a polar aprotic solvent can effectively deliver a biological active compound.

According to a first aspect, there is provided a carrier composition for delivery of a biologically active compound comprising a phosphate compound of an electron transfer agent and a polar aprotic solvent.

There is also provided use of a phosphate compound of an electron transfer agent and a polar aprotic solvent in the preparation of a carrier composition for delivery of a biologically active compound.

There is further provided a process for the preparation of a carrier composition for delivery of a biologically active compound which comprises the step of combining a phosphate compound of an electron transfer agent and a polar aprotic solvent.

The electron transfer agent may be an antioxidant or a derivatised compound thereof. In a preferred embodiment the electron transfer agent is a hydroxy chroman, preferably a tocol such as tocopherol or tocotrienol.

The phosphate compounds of tocopherol may be selected from the group consisting of mono-(tocopheryl)phosphate, mono-(tocopheryl)phosphate monosodium salt, mono-(tocopheryl)phosphate disodium salt, di-(tocopheryl)phosphate, di-(tocopheryl)phosphate monosodium salt, or a mixture thereof.

When the carrier composition comprises a mixture of a mono-(tocopheryl)phosphate to a di-(tocopheryl)phosphate, the ratio may be at least 2:1, within the range of about 4:1 to about 1:4, or within the range of about 6:4 to about 8:2. In some embodiments the ratio is about 6:4 or about 8:2.

The carrier composition comprises a phosphate compound of an electron transfer agent in an amount within the range of about 0.01% w/w to about 20% w/w, about 0.01% w/w to about 10% w/w, about 0.01% w/w to about 5% w/w, or about 0.05% w/w to about 2% w/w, of the total concentration of the carrier composition. In some embodiments the carrier composition comprises a phosphate compound of an electron transfer agent in an amount of about 0.1% w/w, about 1% w/w, or about 5% w/w, of the total concentration of the carrier composition.

The polar aprotic solvent may be selected from the group consisting of N,N-dimethyl-formamide (DMF), N-methyl-2-pyrrolidone (NMP), dimethylsulfoxide (DMSO), N,N-dimethylacetamide (DMAC), dimethyl sulfoxide, dioxane hexamethylphosphorotriamide, tetrahydrofuran, propylene carbonate, gamma-butyrolacetone, monomethyl ether acetate, ethyl lactate, and 1,3 dimethyl-2-imidazolidinone (dimethyl isorbide, or DMI).

The polar aprotic solvent may also be selected from the group consisting of isopropyl myristate, isopropyl palmitate, isopropyl isostearate, diisopropyl adipate, diisopropyl dimerate, maleated soybean oil, octyl palmitate, cetyl lactate, glycerine, polypropylene glycol, caprylyl glycol, squalene, Bisabolol, benzylalcohol, cetyl ricinoleate, cetyl acetate, wheat germ glycerides, myristyl lactate, decyl oleate, isopropyl lanolate, pentaerythrityl tetrastearate, neopentylglycol dicaprylate/dicaprate, isononyl isononanoate, isotridecyl isononanoate, myristyl myristate, octyl dodecanol, and octyl hydroxystearate.

The carrier composition may have a polar aprotic solvent concentration of from about 0.05% w/w up to about 50% w/w, up to about 40% w/w, up to about 30% w/w, up to about 20% w/w, up to about 10% w/w, up to about 5% w/w, up to about 3% w/w, up to about 2% w/w, or up to about 1% w/w.

In a second aspect, there is provided a formulation comprising the carrier composition and a biologically active compound.

There is also provided a process for the preparation of the formulation which comprises the step of adding a biologically active compound to the carrier composition.

The biologically active compound may be a pharmaceutical or pharmaceutically acceptable derivative thereof, a nutraceutical or nutraceutically acceptable derivative thereof, or a cosmeceutical or cosmeceutically acceptable derivatives thereof.

The biologically active compound may be present in an amount of from about 0.001% w/w up to about 15% w/w, up to about 10% w/w, up to about 5% w/w, up to about 2% w/w, or up to about 1% w/w, or within the range of from about 0.001% w/w up to about 0.05% w/w, up to about 1% w/w, up to about 2% w/w, or up to about 5% w/w, of the total concentration of the carrier composition.

In a third aspect, there is provided use of the carrier composition to improve the delivery of the biologically active compound. The carrier composition can improve and/or enable the delivery of a biological active compound, particularly via enteral or parental routes of administration.

The carrier composition may also improve the bioavailability of a biologically active compound in a subject.

A carrier composition of the present invention can also be used in a method for treating a subject for a pathological condition, the method comprising administering an effective amount of a biologically active compound in the carrier composition. The pathological conditions include those that can be treated by the biologically active compound formulated with the carrier composition.

In a fourth aspect, there is a provided use of a polar aprotic solvent to increase the solubility and/or stability of the phosphate compound of the electron transfer agent, particularly in a carrier composition.

### DETAILED DESCRIPTION

The present invention relates to a carrier composition comprising a phosphate compound of an electron transfer agent and a polar aprotic solvent. Biologically active compounds formulated with the carrier composition have been shown to have improved properties.

### Phosphate Compound of an Electron Transfer Agent

The term “electron transfer agent” refers to a compound that may be phosphorylated and which, in the non-phosphorylated form, can accept an electron to generate a relatively stable molecular radical or can accept two electrons to allow the compound to participate in a reversible redox system. Examples of electron transfer agents include antioxidants and derivatives thereof.

The term “antioxidant” refers to a molecule capable of slowing or preventing the oxidation of other molecules. Oxidation is a chemical reaction that transfers electrons from a substance to an oxidizing agent. Oxidation reactions can produce free radicals, which start chain reactions that damage cells. Antioxidants terminate these chain reactions by removing free radical intermediates, and inhibit other oxidation reactions by being oxidized themselves. As a result, antioxidants are often reducing agents.

Antioxidants are generally classified into two broad divisions, depending on whether they are soluble in water (hydrophilic) or in lipids (hydrophobic). Ascorbic acid (vitamin C) is an example of a water soluble antioxidant. Carotenes, tocopherol (Vitamin E), retinol (Vitamin A), ubiquinol (the reduced form of coenzyme Q) and calciferol (Vitamin D) are examples of lipid soluble antioxidants.

Carotenes are carotenoids containing no oxygen. Carotenoids are based on carotenes with one or more hydrogen atoms substituted by a hydroxyl group and/or some pairs of hydrogen atoms are substituted by oxygen atoms. The term “hydroxy carotenoids” refers to carotenes substituted with one or more hydroxyl groups. Cryptoxanthin is an example of a hydroxy carotenoid: it is closely related to beta-carotene with only the addition of a hydroxyl group.

Vitamin E exists in eight different forms, namely four tocopherols and four tocotrienols. All feature a chroman ring, with a hydroxyl group that can donate a hydrogen atom to reduce free radicals and a hydrophobic side chain which allows for penetration into biological membranes. Such derivatives of Vitamin E may be classified as “hydroxy chromans”. Both tocopherols and tocotrienols occur in alpha, beta, gamma and delta forms, determined by the number and location of methyl groups on the chroman ring. The tocotrienols differ from the analogous tocopherols by the presence of three double bonds in the hydrophobic side chain. The various forms of Vitamin E are shown by Formula (I):

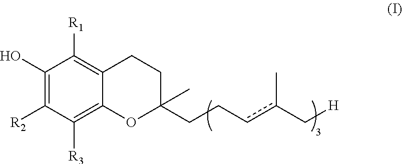

| | R₁ | R₂ | R₃ |
|---|---|---|---|
| α-tocopherol | | | |
| α-tocotrienol | CH₃ | CH₃ | CH₃ |
| β-tocopherol | | | |
| β-tocotrienol | CH₃ | H | CH₃ |
| γ-tocopherol | | | |
| γ-tocotrienol | H | CH₃ | CH₃ |
| δ-tocopherol | | | |
| δ-tocotrienol | H | H | CH₃ |

Retinol belongs to the family of chemical compounds known as retinoids. There are three generations of retinoids. First generation retinoids include retinol, retinal, tretinoin (retinoic acid, Retin-A), isotretinoin and alitretinoin. Second generation retinoids include etretinate and its metabolite acitretin. Third generation retinoids include tazarotene, bexarotene and adapalene.

Ubiquinol is a benzoquinol and is the reduced form of ubiquinone (coenzyme Q₁₀). Calciferol (Vitamin D) comes in several forms. The two major forms are vitamin D₂ (e.g. ergocalciferol) and vitamin D₃ (e.g. calcitriol, cholecalciferol). The other forms include vitamin D₁ (molecular compound of ergocalciferol with lumisterol, 1:1), vitamin D₄ (22-dihydroergocalciferol) and vitamin D5 (sitocalciferol, made from 7-dehydrositosterol).

Any antioxidant or derivative thereof described herein would be suitable for the present invention. Preferred antioxidants and derivatives thereof are selected from the group consisting of carotenoids, hydroxy chromans, carotenoids, retinoids, benzoquinols and calcitriols. Hydroxy chromans are preferred. Tocols such as a tocopherol, in any form, is most preferred.

The term “phosphate compound” refers to a phosphorylated compound, where a covalent bond is formed between an oxygen atom (typically originating from a hydroxyl group) of the compound and the phosphorous atom of a phosphate group (PO₄): in this context, the compound is an electron transfer agent.

The phosphate compound may be a phosphate mono-ester, phosphate di-ester, is phosphate tri-ester, pyrophosphate mono-ester, pyrophosphate di-ester, or a salt or derivative thereof, or a mixture thereof. The di- and tri-esters may comprise the same electron transfer agent or different electron transfer agents.

The “salts” include metal salts such as alkali or alkaline earth metal salts, for example sodium, magnesium, potassium and calcium salts. Sodium and potassium salts are preferred.

The “derivatives” include phosphate compounds where one or more phosphate protons are replaced by a substituent. Some non-limiting examples of derivatives include phosphatidyl derivatives where a phosphate proton is substituted with an amino-alkyl group, sugar derivatives where a phosphate proton is substituted with a sugar such as glucose.

The term “amino-alkyl group” refers to a group comprising an amino (—NH₂) group and an alkyl group. The term “alkyl” refers to straight chain, branched chain or cyclic hydrocarbon groups having from 1 to 8 carbon atoms. Examples include methyl, ethyl, propyl, isopropyl, butyl, isobutyl, sec-butyl, tert-butyl, pentyl, hexyl, cyclohexyl, heptyl, and octyl. Phosphatidyl choline derivatives are most preferred.

When the electron transfer agent is tocopherol, examples of phosphate compounds of tocopherol include but are not limited to mono-(tocopheryl)phosphate, mono-(tocopheryl)phosphate monosodium salt, mono-(tocopheryl)phosphate disodium salt, di-(tocopheryl)phosphate, di-(tocopheryl)phosphate monosodium salt, or a mixture thereof. These phosphate compounds may be derived from the alpha, beta, gamma or delta form of tocopherol, or a combination thereof.

In some embodiments, it may be desirable, particularly for delivery of nutraceutical and cosmeceutical actives, to use a non-neutralised form of the phosphate compounds of tocopherol. These embodiments provide a carrier comprising non-neutralised tocopheryl phosphate and a polar aprotic solvent. The pH of the non-neutralised tocopheryl phosphate may be in the range of about 2 to about 4, or about 2 to about 3. In some embodiments, the pH of the non-neutralised tocopheryl phosphate is about 2 or about 3.

When the carrier composition contains a mixture of a mono-phosphate ester and a di-phosphate ester, for example a mono-(tocopheryl)phosphate and di-(tocopheryl)phosphate (which may be referred to as “TPM” herein), the ratio may be at least 2:1, within the range of about 4:1 to about 1:4, or within the range of about 6:4 to about 8:2. In some embodiments the ratio may be about 6:4 or about 8:2.

The carrier composition comprises a phosphate compound of an electron transfer agent in an amount within the range of about 0.01% w/w to about 20% w/w, about 0.01% w/w to about 10% w/w, about 0.01% w/w to about 5% w/w, or about 0.05% w/w to about 2% w/w, of the total concentration of the carrier composition. In some embodiments the carrier composition comprises a phosphate compound of an electron transfer agent in an amount of about 0.1% w/w, about 1% w/w, or about 5% w/w, of the total concentration of the carrier composition.

### Polar Aprotic Solvent

In chemistry, solvents may be grouped into non-polar aprotic, polar aprotic, and polar protic solvents, ordered by increasing polarity. The polarity of a solvent determines what type of compounds it is able to dissolve and with what other solvents or liquid compounds it is miscible. Generally, polar solvents dissolve polar compounds best and non-polar solvents dissolve non-polar compounds best, i.e. “like dissolves like”.

The carrier composition comprises a polar aprotic solvent.

Examples of polar aprotic solvents include, but are not limited to, N,N-dimethyl-formamide (DMF), N-methyl-2-pyrrolidone (NMP), dimethylsulfoxide (DMSO), N,N-dimethylacetamide (DMAC), dimethyl sulfoxide, dioxane hexamethylphosphorotriamide, tetrahydrofuran, propylene carbonate, gamma-butyrolacetone, monomethyl ether acetate, ethyl lactate, and 1,3 dimethyl-2-imidazolidinone (dimethyl isorbide, or DMI).

Polar aprotic solvents may also be selected from the family of organic liquids described as “emollients”. Emollients possess a softening or soothing effect, especially when applied to body areas, such as the skin and mucosal surfaces. Examples of suitable emollients include isopropyl myristate, isopropyl palmitate, isopropyl isostearate, diisopropyl adipate, diisopropyl dimerate, maleated soybean oil, octyl palmitate, cetyl lactate, glycerine, polypropylene glycol, caprylyl glycol, squalene, Bisabolol, benzylalcohol, cetyl ricinoleate, cetyl acetate, wheat germ glycerides, myristyl lactate, decyl oleate, isopropyl lanolate, pentaerythrityl tetrastearate, neopentylglycol dicaprylate/dicaprate, isononyl isononanoate, isotridecyl isononanoate, myristyl myristate, octyl dodecanol, and octyl hydroxystearate.

The carrier composition may comprise only one polar aprotic solvent; however, a mixture or combination of polar aprotic solvent can also be used. For the avoidance of any doubt, it is to be noted that the singular forms “a”, “an” and “the” should be read as encompassing plural forms, unless the context clearly indicates otherwise.

In preferred embodiments, the polar aprotic solvent would have a relatively high hydrophobicity level, while still being miscible in water. Particularly preferred, are those that have ability to increase the solubility of lipid soluble molecules, such as tocopheryl phosphate, in an aqueous environment.

While the polar aprotic solvent concentration in the carrier composition will vary depending on the polar aprotic solvent used and/or the biologically active compound to be formulated with the carrier composition, it has been surprisingly found that only relatively small amount will be necessary to achieve the desired result. However, the carrier composition may have a polar aprotic solvent concentration of up to about 50% w/w. Accordingly, the carrier composition may have a polar aprotic solvent concentration of from about 0.05% w/w up to about 50% w/w, up to about 40% w/w, up to about 30% w/w, up to about 20% w/w, up to about 10% w/w, up to about 5% w/w, up to about 3% w/w, up to about 2% w/w, or up to about 1% w/w.

### Biologically Active Compound

The term “biologically active compound” refers to any chemical substance that has a biological effect in humans or animals for medical, therapeutic, cosmetic and veterinary purposes, and encompasses pharmaceuticals including drugs, cosmeceuticals, nutraceuticals, and nutritional agents. It will be appreciated that some of biologically active compounds can be classified in more than one of these classes.

A wide range of biologically active compounds may be delivered with the carrier composition of the present invention. Examples include, but are not limited to, cardiovascular drugs, in particular antihypertensive agents (e.g. calcium channel blockers or calcium antagonists) and antiarrhythmic agents; congestive heart-failure pharmaceuticals; inotropic agents; vasodilators; ACE inhibitors; diuretics; carbonic anhydrase inhibitors; cardiac glycosides; phosphodiesterase inhibitors; a blockers; β-blockers; sodium channel blockers; potassium channel blockers; β-adrenergic agonists; platelet inhibitors; angiotensin II antagonists; anticoagulants; thrombolytic agents; treatments for bleeding; treatments for anaemia; thrombin inhibitors; antiparasitic agents; antibacterial agents; insulin; human growth hormone and peptides; vaccines; antiinflammatory agents, in particular non-steroidal antiinflammatory agents (NSAIDs), more particularly COX-2 inhibitors; steroidal antiinflammatory agents; prophylactic antiinflammatory agents; antiglaucoma agents; mast cell stabilisers; mydriatics; agents affecting the respiratory system; allergic rhinitis pharmaceuticals; alpha-adrenergic agonists; corticosteroids; chronic obstructive pulmonary disease pharmaceuticals; xanthine-oxidase inhibitors; antiarthritis agents; gout treatments; autacoids and autacoid antagonists; antimycobacterial agents; antifungal agents; antiprotozoal agents; anthelmintic agents; antiviral agents especially for respiratory, herpes, cyto-megalovirus, human immunodeficiency virus and hepatitis infections; treatments for leukemia and kaposi's sarcoma; pain management agents in particular anaesthetics and analgesics, opioids including opioid receptor agonists, opioid receptor partial agonists, opioid antagonist or opioid receptor mixed agonist-antagonists; neuroleptics; sympathomimetic pharmaceuticals; adrenergic agonists; drugs affecting neurotransmitter uptake or release; anticholinergic pharmaceuticals; antihaemorrhoid treatments; agents to prevent or treat radiation or chemotherapeutic effects; liopgenisis drugs; fat reducing treatments; anti-obesity peptides; antiobesity agents such as lipase inhibitors; sympathomimetic agents; treatments for gastric ulcers and inflammation such as proton pump inhibitors; prostaglandins; VEGF inhibitors; antihyperlipidemic agents, in particular statins; drugs that affect the central nervous system (CNS) such as antipsychotic, antiepileptic and antiseizure drugs (anticonvulsants), psychoactive drugs, stimulants, antianxiety and hypnotic drugs, antidepressant drugs; antiparkinson's pharmaceuticals; hormones and fragments thereof such as sex hormones; growth hormone antagonists; gonadotropin releasing hormones and analogues thereof; steroid hormones and their antagonists; selective estrogen modulators; growth factors; antidiabetic pharmaceuticals such as insulin, insulin fragments, insulin analogues, glucagon-like peptides and hypoglycaemic agents; H1, H2, H3 and H4 antihistamines; peptide, protein, polypeptide, nucleic acids and oligonucleotide pharmaceuticals; analogues, fragments and variants of natural proteins, polypeptides, oligonucleotides and nucleic acids and such like compounds; agents used to treat migraine headaches; asthma pharmaceuticals; cholinergic antagonists; glucocorticoids; androgens; antiandrogens; inhibitors of adrenocorticoid biosynthesis; osteoporosis treatments such as biphosphonates; antithyroid pharmaceuticals; suncreens, sun protectants and filters; cytokine agonists; cytokine antagonists; anticancer drugs; antialzheimer drugs; HMGCoA reductase inhibitors; fibrates; cholesterol absorption inhibitors; HDL cholesterol elevating agents; triglyceride reducing agents; antiageing or antiwrinkle agents; precursor molcules for the generation of hormones; proteins such as collagen and elastin; antibacterial agents; anti acne agents; antioxidants; hair treatments and skin whitening agents; suncreens, sun protectants and filters; variants of human apolipoprotein; precursor molecules for generation of hormones; proteins and peptides thereof; amino acids; plant extracts such as grape seed extract; DHEA; isoflavones; nutritional agents including vitamins, phytosterols and iridoid gylcosides, sesquiterpene lactones, terpenes, phenolic glycosides, triterpenes, hydroquinone derivatives, phenylalkanones; antioxidants such as retinol and other retinoids including retinoic acid and co enzyme Q10; omega-3-fatty acids; glucosamine; nucleic acids, oligonucleotides, antisense pharmaceuticals; enzymes; cytokines; cytokine analogues; cytokine agonists; cytokine antagonists; immunoglobulins; antibodies; antibody pharmaceuticals; gene therapies; lipoproteins; erythropoietin; vaccines; small and large molecule therapeutic agents for the treatment, or prevention of human and animal diseases such as allergy/asthma, arthritis, cancer, diabetes, growth impairment, cardiovascular diseases, inflammation, immunological disorders, baldness, pain, ophthalmological diseases, epilepsy, gynaecological disorders, CNS diseases, viral infections, bacterial infections, parasitic infections, GI diseases, obesity, and haemological diseases.

Some specific non-limiting examples of suitable biologically active compounds include:

### Anaesthetics:

including amino-ester and amino-amide anaesthetics such as benzocaine, chloroprocaine, cocaine, reserpine, guanethidine, cyclomethycaine, dimethocaine/larocaine, propoxycaine, procaine/novocaine, proparacaine, tetracaine/amethocaine; articaine, bupivacaine, carticaine, cinchocaine/dibucaine, etidocaine, levobupivacaine, lidocaine/lignocaine, mepivacaine, piperocaine, prilocalne, ropivacaine, trimecaine, propofol, halothane, enflurane barbiturates, benzodiazepines, neostigmine and ketamine

### Alkylating Agents:

including carmustine, cyclophosphamide, ifosfamide, streptozotocin and mechlorethamine

### Calcium Channel Blockers:

including amlodipine, aranidipine, azelnidipine, barnidipine, benidipine, cilnidipine, clevidipine, cronidipine, darodipine, dexniguldipine, efonidipine, elnadipine, elgodipine, felodipine, flordipine, furnidipine, iganidipine, isradipine, lacidipine, lemildipine, lercanidipine, manidipine, mesuldipine, nicardipine, nifedipine, niludipine, nilvadipine, nimodipine, nisoldipine, nitrendipine, olradipine, oxodipine, palonidipine, pranidipine, sagandipine, sornidipine, teludipine, tiamdipine, trombodipine, watanidipine, verapamil, gallopamil, benzothiazepine, diltiazem, mibefradil, bepridil, fluspirilene and fendiline

### Antiarrhythmic and Antiangina Agents:

including amiodarone, disopyramide, flecamide acetate, quinidine sulphate, nitroglycerine, ranolazine, amiodarone, isosorbide and alteplase

### Antibacterial, Antibiotic and Antiacne Agents:

including amoxicillin, ampicillin, azithromycin, benethamine penicillin, bleomycin, benzoyl peroxide, cinoxacin, chloramphenicol, daunorubicin, plicamycin, fluoroquinolones, ciprofloxacin, clarithromycin, clindamycin, clindesse, clofazimine, chlorohexidine gluconate, cloxacillin, demeclocycline, doxycycline, erythromycin, ethionamide, imipenem, indomethacin, lymocycline, minocycline, nalidixic acid, nitrofurantoin, penicillin, rifampicin, spiramycin, sodium sulfacetamide, sulphabenzamide, sulphadoxine, sulphamerazine, sulphacetamide, sulphadiazine, sulphafurazole, sulphamethoxazole, sulphapyridine, tetracycline, cephalexin, cefdinir, triclosan, ofloxacin, vancocin, glyburide, mupirocin, cefprozil, cefuroxime axetil, norfloxacin, isoniazid, lupulone, D-penicillamine, levofloxacin, gatifoxacin, and trimethoprim

### Anticancer:

including doxorubicin, 6-thioguanine, paclitaxel, docetaxel, camptothecin, megestrol acetate, navelbine, cytarabine, fludarabine, 6-mercaptopurine, 5-fluorouracil, teniposide, vinblastine, vincristine, cisplatin, colchicine, carboplatin, procarbazine and etopside

### Antidepressants, Antipsychotics and Antianxiety:

including alprazolam, amoxapine, bentazepam, bromazepam, clorazipine, clobazam, clotiazepam, diazepam, lorazepam, flunitrazepam, flurazepam, lormetazepam, medazepam, nitrazepam, oxazepam, temazepam, maprotiline, mianserin, nortriptyline, risperidone, sertraline, trazodone, baloperidol, trimipramine maleate fluoxetine, ondansetron, midazolam, chlorpromazine, haloperidol, triazolam, clozapine, fluopromazine, fluphenazine decanoate, fluanisone, perphenazine, pimozide, prochlorperazine, sulpiride, thioridazine, paroxitine, citalopram, bupropion, phenelzine, olanzapine, divalproex sodium and venlafaxine

### Opioids:

including opioid receptor agonists and antagonists, compounds which exhibit mixed agonist/antagonist activity and compounds which exhibit partial agonist activity, including morphine, depomorphine, etorphine, diacetylmorphine, hydromorphone, oxymorphone, levorphanol, methadone, levomethadyl, meperidine, fentanyl, sufentanyl, alfentanil, codeine, hydrocodone, oxycodone, thebaine, desomorphine, nicomorphine, dipropanoylmorphine, benzylmorphine, ethylmorphine, pethidine, methadone, tramadol, dextropropoxyphene; naloxone and naltrexone; buprenorphine, nalbuphine, butorphanol, pentazocine and ethylketocyclazocine

### Tricyclics:

including azothiopine, amitriptyline, famotidine, promethazine, paroxatine, oxcarbazapine and mertazapine

### Antidiabetics:

including acetohexamide, chlorpropamide, glibenclaraide, gliclazide, glipizide, metformin, tolazamide, glyburide, glimepiride and tolbutamide

### Antiepileptics:

including beclamide, carbamazepine, gapapentin, tiagabine, vigabatrin, topiramate, clonazepam, ethotoin, methoin, methsuximide, methylphenobarbitone, oxcarbazepine, paramethadione, phenacemide, phenobarbitone, phenyloin, phensuximide, primidone, sulthiamine, phenyloin sodium, nirofurantoin monohydrate, gabapentin, lamotrigine, zonisamide, ethosuximide and valproic acid

### Hypnotics/Sedatives and Muscle Relaxants:

including zolpidem tartrate, amylobarbitone, barbitone, butobarbitone, pentobarbitone, brotizolam, carbromal, chlordiazepoxide, chlormethiazole, ethinamate, meprobamate, methaqualome, cyclobenzaprene, cyclobenzaprine, tizanidine, baclofen, butalbital, zopiclone, atracurium, tubocurarine and phenobarbital

### Antifungal, Antiprotazoal and Antiparasitic Agents:

including amphotericin, butoconazole nitrate, clotrimazole, econazole nitrate, fluconazole, flucytosine, griseofulvin, itraconazole, ketoconazole, miconazole, natamycin, nystatin, sulconazole nitrate, terconazole, tioconazole and undecenoic acid; benznidazole, clioquinol, decoquinate, diiodohydroxyquinoline, diloxanide furoate, dinitolmide, furzolidone, metronidazole, nimorazole, nitrofurazone, ornidazole, terbinafine, clotrimazole, chloroquine, mefloquine, itraconazole, pyrimethamine, praziquantel, quinacrine, mebendazole and timidazole

### Antihypertensive and Cardiac Therapeutic Agents:

including candesartan, hydralazine, clonidine, triamterene, felodipine, gemfibrozil, fenofibrate, nifedical, prazosin, mecamylamine, doxazosin, dobutamine and cilexetil

### Antimigraine Agents:

including dihydroergotamine mesylate, ergotamine tartrate, methysergide maleate, pizotifen maleate and sumatriptan succinate

### Antimuscarinic Agents:

including atropine, benzhexyl, biperiden, ethopropazine, hyoscyamine, mepenzolate bromide, oxybutynin, oxyphencylcimine and tropicamide

### Antineoplastic Agents (or Immunosuppressants):

including aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine, tamoxifen citrate, testolactone, tacrolimus, mercaptopurine and sirolimus

### Antiparkinsonian Agents:

including bromocriptine mesylate, levodopa, tolcapone, ropinirole, bromocriptine, hypoglycaemic agents such as sulfonylureas, biguanides, α-glucosidase inhibitors, thaiazolidinediones, cabergoline, carbidopa and lysuride maleate

### Antithyroid Agents:

including carbimazole and propylthiouracil

### Antiviral Drugs:

including amantadine, retinovir, cidofovir, acyclovir, famciclovir, ribavirin, amprenavir, indinavirm, rimantadine and efavirenz, penciclovir, ganciclovir, vidarabine, abacavir, adefovir, apmrenavir, delavirdine, didanosine, stavudine, zalcitabine, zidovudine, enfuvirtide and interferon

### Cardiac Inotropic Agents:

including aminone, milrinone, digitoxin, digoxin, enoximone, lanatoside C and medigoxin

### Hypo and Hyper Lipidemic Agents:

including fenofibrate, clofibrate, probucol, ezetimibe and torcetrapib

### Antiinflammatory:

including meoxicam, triamcinolone, cromolyn, nedocromil, hydroxychloroquine, montelukast, zileuton, zafirlukast and meloxicam

### Antihistamine:

including fexofenadine, chloral hydrate, hydroxyzine, promethazine, cetirazine, cimetidine, clyclizine, meclizine, dimenhydrinate, loratadine, nizatadine and promethazine

### Antiulcer:

including omeprazole, lansoprazole, pantoprazole and ranitidine

### Diuretics:

including hydrochlorothiazide, amiloride, acetazolamide, furosemide and torsemide

### NSAIDs:

including arylalkanoic acid sub-group of class which includes diclofenac, aceclofenac, acemetacin, alclofenac, bromfenac, etodolac, indometacin, indometacin farnesil, nabumetone, oxametacin, proglumetacin, sulindac and tolmetin; 2-arylpropionic acid (profens) sub-group of class which includes alminoprofen, benoxaprofen, carprofen, dexibuprofen, dexketoprofen, fenbufen, fenoprofen, flunoxaprofen, flurbiprofen, ibuprofen, ibuproxam, indoprofen, ketoprofen, ketorolac, loxoprofen, miroprofen, naproxen, oxaprozin, pirprofen, suprofen, tarenflurbil and tiaprofenic acid; and N-arylanthranilic acid (fenamic acid) sub-group of class which includes flufenamic acid, meclofenamic acid, mefenamic acid and tolfenamic acid; tromethamine, celecoxib, nepafenac, aspirin, rofecoxib, naproxen, sulindac, piroxicam, pheylbutazone, tolmetin, indomethacin, acetominophen (paracetamol), tramadol and propoxyphene

### Retinoids:

including first generation retinoids such as retinol, retinal, tretinoin (retinoic acid, Retin-A), isotretinoin and alitretinoin; second generation retinoids such as etretinate and its metabolite acitretin; third generation retinoids such as tazarotene, bexarotene and adapalene

### Hormones and Steroids:

including adrenocorticotrophic hormone (ACTH), antidiruetic hormone (vasopressin), atrial-nartreuretic factor (ANF), atrial-nartreuretic peptide (ANP), beclomethasone, cortisone, scopolamine, dopamine, epinephrine, catecholamines, cholecystokinin, clomiphene citrate, danazol, dexamethasone, diethylstilbestrol (DES), ethinyl estradiol, fludrocortison, finasteride, follicle stimulating hormone, gastrin, hydroxyprogesterone, growth hormone, insulin, leptin, luteinizing hormone, medroxyprogesterone acetate, mestranol, quinestrol, methyltestosterone, nandrolone, norethindrone, norethisterone, norgestrel, estradiol, conjugated oestrogens, oxandrolone, oxytocin, prednisone, progesterone, prolactin, protogalndins, somatostatin, stanozolol, stibestrol, thyroxine, prednisolone phosphate, triamcinolone, mifepristone acetonide, budesonide, levothyroxine, testosterone, testosterone cypionate, fluoxymesterone, flutamide, mometasone furoate, cyproterone, fluoromethalone, goserelin, leuprolide, calcitonin, halobetasol, hydrocortisol and tibolone

### Statins and Derivatives:

including atorvastatin, fluvastatin, lovastatin, nystatin, rosuvastatin, pravastatin, orlistat and simvastatin

### Stimulants:

including amphetamine, phentermine, tyramine, ephedrine, metaraminol, phenylephrine, dexamphetamine, dexfenfluramine, fenfluramine, nicotine, caffeine and mazindol

### Vasocontrictors:

including desmopressin

### Vasodilitors:

including carvedilol, terazosin, phentolamine and menthol

### Antialzheimers:

including levetiracetam, levitiracetam and donepezil

### ACE Inhibitors:

including benzapril, enalapril, ramipril, fosinopril sodium, lisinopril, minoxidil, isosorbide, rampril and quinapril

### Beta Adrenoreceptor Antagonists:

including atenolol, timolol, pindolol, propanolol hydrochloride, bisoprolol, esmolol, metoprolol succinate, metoprolol and metoprolol tartrate

### Angiotensin II Antagonists:

including losartan

### Platelet Inhibitors:

including abciximab, clopidrogel, tirofiban and aspirin

### Alcohols and Phenols:

including tramadol, tramadol hydrochloride, allopurinol, calcitriol, cilostazol, soltalol, urasodiol bromperidol, droperidol, flupenthixol decanoate, albuterol, albuterol sulphate, carisoprodol, chlobetasol, ropinirol, labetalol, and methocarbamol

### Ketones and Esters:

including amioderone, fluticasone, spironolactone, prednisone, triazodone, desoximetasone, methyl prednisdone, benzonatate nabumetone and buspirone

### Antiemetics:

including metoclopramide

### Ocular Treatments:

including dorzolamide, brimonidine, olopatadine, cyclopentolate, pilocarpine and echothiophate

### Anticoagulant and Antithrombitic Agents:

including warfarin, enoxaparin and lepirudin

### Treatments for Gout:

including probenecid and sulfinpyrazone

### COPD and Asthma Treatments:

including ipratropium

### Treatments for Osteoporosis:

including raloxifene, pamidronate and risedronate

### Cosmetic Peptides:

including acetyl hexapeptide-3, acetyl hexapeptide-8, acetyl octapeptide and l-carnosine

### Vaccines:

including vaccines comprising toxoids (inactivated toxic compounds); proteins, protein subunits and polypeptides; polynucleotides such as DNA and RNA; conjugates; adjuvants such as saponins, virosomes, inorganic and organic adjuvants, for example zostavax

### Nutraceutical and Cosmeceutical Actives:

including coenzyme Q₁₀ (or ubiquinone), ubiquinol or resveratrol; a carotenoid such as α, β, or γ-carotene, lycopene, lutein, zeaxanthin and astaxanthin; a phytonutrient, such as lycopene, lutein and seaxanthin; an unsaturated fatty acid such as linoleic acid, conjugated linoleic acid, linolenic acid, omega-3 fatty acids including but not limited to docosahexaenoic acid (DHA) and eicosapentaeonic acid (EPA) and their glycerol-esters; fat-soluble vitamins including vitamin D (D2, D3 and their derivatives), vitamin E (α, β, γ, δ-tocopherols, or α, β, γ, δ-tocotrienols), vitamin A (retinol, retinal, retinoic acid and derivatives), vitamin K (K₁, K₂, K₃ and their derivatives) capric/caprylic triglycerides, folic acid, iron, niacin, glyceryl linoleate, omega 6 fatty acids, vitamin F, selenium, cyanocobalamin, aloe vera, beta glucan, bisabolol, camellia thea (green tea) extract, capric/caprylic triglycerides, centella asiatica (gotu cola) extract, cetearyl olivate, chlorophyll, citrus sinensis (orange) oil, cocoyl proline, dicapryl ether, disodium lauriminodipropionate tocopheryl phosphates (vitamin E phosphates), glycerin, glyceryl oleate, glycyrrhiza glabra (licorice) root extract, hamamelis virgiana (witch hazel) extract, lactic acid, lecithin, lutein, macadamia integrifolia (macadamia) seed oil, matricaria chamomilla (chamomile) extract, oenothera biennis (evening primrose) oil, olea europaea (olive) leaf extract, rice bran oil, persea gratissima (avocado) oil, polygonum multiflorum extract, pomegranate sterols, resveratrol, rosa eglanteria (rose hip) oil, santalum spicatum (sandalwood) oil, titanium dioxide, folic acid, glycerin, glyceryl linoleate (omega 6 fatty acids vitamin F), vitamin A palmitate, vitis vinifera (grapeseed) oil, halobetasol, adenosine, adenosine triphosphate, alpha hydroxy acid, allantoin, hyaluronic acid and derivatives, isolutrol, tranexamic acid, glycolic acid, arginine, ascorbyl glucosamine, ascorbyl palmitate, salicylic acid, carnosic acid, alpha lipoic acid, gamma linolenic acid (GLA), panthenol, retinyl propionate, retinyl pamitate, furfuryladenine, retinaldehyde, copper pepetides, idebenone, dimethylaminoethanol (DMAE), niacinamide, beta-glucan, palmitoyl pentapeptide-4, palmitoyl oligopeptide/tetrapetide-7, ethocyn, ceramides, phenylalanine, glucuronolactone, L-carnitine, hydroxylapetite, palmitoyl tripetide-3, forskolin, zinc oxide, α-bisabolol, eugenol, silybin, soy isoflavones, aucubin, catalpol, pseudoguaianolide from Arnica chamissonis, rosmarinic acid, rosmanol, salicylates for example salicin, saligenin and salicyclic acid, taxasterol, α-lactucerol, isolactucerol, taraxacoside, ceremides, arbutin, gingerols, shagaols, hypercin, elastin, collagen and peptides thereof.

Particularly preferred biologically active compounds include lidocaine, diclofenac, ketoralac, prilocalne, halobetasol, hydrocortisol and combinations thereof.

It is to be understood that pharmaceutically, nutraceutically or cosmeceutically acceptable derivatives of biologically active compounds are included within the scope of the present invention.

The term “pharmaceutically, nutraceutically or cosmeceutically acceptable derivatives” includes, but is not limited to, pharmaceutically, nutraceutically or cosmeceutically acceptable salts, esters, salts of such esters, ethers, or any other derivative including prodrugs and metabolites, which upon administration to a subject (e.g. patient, human or animal) in need is capable of providing, directly or indirectly, a biologically active compound as otherwise described herein.

As used herein, the term “pharmaceutically, nutraceutically or cosmeceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically, nutraceutically or cosmeceutically acceptable salts are well known in the art. For example, S. M. Berge, et al. describe pharmaceutically, nutraceutically or cosmeceutically acceptable salts in detail in J. Pharmaceutical Sciences, 66:1-19, 1977. Examples of pharmaceutically, nutraceutically or cosmeceutically acceptable nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically, nutraceutically or cosmeceutically acceptable ester” refers to esters which are hydrolysed in vivo and include those that break down readily in the human body to leave the parent compound or a salt thereof. Suitable ester groups include, for example, those derived from pharmaceutically, nutraceutically or cosmeceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

The term “pharmaceutically, nutraceutically or cosmeceutically acceptable prodrugs” as used herein refers to those prodrugs of the biologically active compounds which are, within the scope of sound medical judgment, suitable for use in contact with the tissues of a subject with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987.

A biologically active compound may be present in any amount necessary to achieve the desired biological effect. Typically, the biologically active compound will be present in an amount of from about 0.001% w/w up to about 15% w/w, up to about 10% w/w, up to about 5% w/w, up to about 2% w/w, up to about 1% w/w, or within the range of from about 0.001% w/w up to about 0.05% w/w, up to about 0.1% w/w, up to about 1% w/w, up to about 2% w/w, up to about 5% w/w, or about 1% w/w, about 2% w/w or about 5% w/w, of the total concentration of the carrier composition.

### Advantages

It has been surprisingly found that the presence of a polar aprotic solvent can increase the solubility of the phosphate compound of the electron transfer agent compared to carrier compositions comprising a phosphate compound of the electron transfer agent with other kinds of solvents such as ethanol which is a polar protic solvent, or surfactants which have well defined polar and non-polar regions.

It has also been found that the polar aprotic solvent can increase the stability of a phosphate compound of an electron transfer agent, particularly when used as a carrier composition for a biologically active compound.

Accordingly, the carrier compositions of the present invention effectively enable:

(a) the content of the phosphate compound of the electron transfer agent to be increased, when necessary. An increased amount of the phosphate compound of the electron transfer agent may increase the effective penetration of the biologically active compound.

(b) the polar aprotic solvent concentration to be decreased relative to the aqueous phase. This is beneficial to the structural fidelity of hydrophilic molecules (like proteins) that can denature and/or precipitate in the presence of organic solvents. The relative increase in the aqueous phase also enables an increase the concentration of is biologically active compounds that are hydrophilic that would otherwise have poorer solubility in a comparative ethanolic formulation.

(c) a potentially safer and easier working environment because polar aprotic solvents are non-flammable.

(d) a more stable solvent solution for storage of the phosphate compounds of electron transfer agents.

Accordingly, a carrier composition of the present invention can improve the delivery of a biological active compound, particularly those administered via enteral or parental routes. The carrier composition may also enable delivery of a biological active compound via enteral or parental routes of administration when previously this was not readily possible.

A carrier composition of the present invention may also improve the bioavailability of a biologically active compound in a subject.

A carrier composition of the present invention can also be used in a method for treating a subject for a pathological condition, the method comprising administering an effective amount of a biologically active compound in a carrier composition of the present invention. The carrier composition may also be used to deliver a biologically active compound to treat a pathological condition in a subject. The pathological conditions include those that can be treated by the biologically active compound formulated with the carrier composition.

Generally, the term “treating” means affecting a subject, tissue or cell to obtain a desired pharmacological and/or physiological effect and includes: (a) inhibiting the viral infection or RSV disease, such as by arresting its development or further development; (b) relieving or ameliorating the effects of the viral infection or RSV disease, such as by causing regression of the effects of the viral infection or RSV disease; (c) reducing the incidence of the viral infection or RSV disease or (d) preventing the infection or disease from occurring in a subject, tissue or cell predisposed to the viral infection or RSV disease or at risk thereof, but has not yet been diagnosed with a protective pharmacological and/or physiological effect so that the viral infection or RSV disease does not develop or occur in the subject, tissue or cell.

The term “subject” refers to any animal, in particular mammals such as humans, having a disease which requires treatment with the compound of formula (I).

The term “administering” should be understood to mean providing a compound or pharmaceutical composition of the invention to a subject suffering from or at risk of the disease or condition to be treated or prevented.

The term “therapeutically effective amount” refers to the amount of the compound of formula (I) that will elicit the biological or medical response of a subject, tissue or cell that is being sought by the researcher, veterinarian, medical doctor or other clinician.

### Routes of Administration

A biologically active compound may be delivered by any route of administration.

Routes of administration can broadly be divided into a three categories by effect, namely, “topical” where the desired effect is local, so the substance is applied directly where its action is desired, “enteral” where the desired effect is systemic (non-local) so the substance is given via the digestive tract, and “parenteral” where the desired effect is systemic, so the substance is given by routes other than the digestive tract.

The U.S. Food and Drug Administration recognise 111 distinct routes of administration. The following is a non-limiting list of examples of routes of administration.

Examples of topical routes of administration having a local effect include epicutaneous (onto the skin) and intravitreal (onto the eye).

Examples of enteral routes of administration having a systemic (non-local) effect include any form of administration that involves any part of the gastrointestinal tract, such as oral (into the mouth), intranasal (into the nose), rectal (into the rectum), and vaginal (into the vagina).

Examples of parenteral routes of administration by injection, infusion or diffusion having a systemic effect include intravenous (into a vein), intraarterial (into an artery), intramuscular (into a muscle), intracardiac (into the heart), subcutaneous (under the skin), percutaneous (via needle-puncture into the skin), intradermal (into the skin itself), intrathecal (into the spinal canal), intraperitoneal (infusion or injection into the peritoneum), intravesical infusion (infusion into the urinary bladder), epidural (injection or infusion into the epidural space), transdermal or transcutaneous (diffusion through the intact skin), transmucosal (diffusion through a mucous membrane), insufflation (diffusion through the nose), inhalational (diffusion through the mouth), sublingual (under the tongue), and buccal (absorbed through cheek near gumline).

As the presence of a polar aprotic solvent can increase the solubility of the phosphate compound of the electron transfer agent, which in turn can increase the effective penetration of a biologically active compound, parenteral routes of administration are preferred. However, the carrier of the present invention may also be suitable for enteral administration.

Formulations according to the present invention can be in any suitable administration form (see, for example, Pharmaceutics and Pharmacy Practice, J. B. Lippincott Company, Philadelphia, Pa., Banker and Chalmers, eds., pages 238-250 (1982)). The formulations and can be prepared by any methods well known in the art of pharmacy such as described in Remington J. P., The Science and Practice of Pharmacy, ed. A. R. Gennaro, 20^{th} edition, Lippincott, Williams and Wilkins Baltimore, Md. (2000). Such methods include the step of bringing into association the biologically active compound with the carrier, and then, if necessary, shaping the formulation into the desired product.

### Dosage Form

Formulations comprising the carrier composition and a biologically active compound may be prepared into any suitable dosage form for enteral or parenteral administration.

A person skilled in the art would readily appreciate what would be a suitable dosage form for enteral or parental administration.

Suitable dosage forms for enteral administration would include but not be limited to capsules, tablets, pills, or specialty tablets such as buccal, sublingual, chewable tablets or orally-disintegrating tablets. Another example of a suitable dosage form would be edible thin films.

Other suitable dosage forms for enteral administration include liquid solutions or suspensions. Suitable liquid solution or suspension dosage forms may be in the form of a drink, such as sports drinks containing electrolytes (e.g. gatorade), or syrup and elixirs. Other suitable liquid solution or suspension dosage forms include nasal delivery solutions and oral suspensions.

The dosage form for enteral administration may also be a powder or solid crystal, which can be either dissolved or suspended in a liquid before administration. Alternatively, the powder may be consumed directly or added to a food or drink product for consumption.

In another example, the dosage form for enteral administration may be a food to which the composition is added before the food is consumed. For example, the food product may for example be a bar such as a health bar, a cereal, bread such as a fortified bread, a cookie, a spread such as butter, a dairy product such cheese or milk, or any other suitable food product.

Where the composition has a disagreeable taste, additives with sufficient flavour to disguise the bad taste may be added to the dosage form (e.g. masking agents).

Examples of suitable dosage forms for parenteral administration include but are not limited to injectables (i.e. solutions, suspensions, emulsions, and dry powders for reconstitution), intramammary infusions, intravaginal delivery systems, reservoir and other patches and implants.

### Preparation of a Carrier Composition

A carrier composition of the present invention may be prepared by a variety of techniques.

One method of preparing the carrier composition involves combining the phosphate compound of the electron transfer agent with the polar aprotic solvent and then adding water. Depending on the solubility and stability of the biologically active compound, it may be dissolved in either the aqueous or solvent phase. Generally, the polar aprotic solvent is heated to a temperature of 30° C. or more and the phosphate compound of the electron transfer agent is dissolved in the polar aprotic solvent. If the biologically active compound is soluble in the polar aprotic solvent, then this is added when the phosphate compound of the electron transfer agent and polar aprotic solvent are combined and the balance of the formulation is made up of water.

The carrier composition may optionally further comprise one or more excipients. A person skilled in the art of the invention would appreciate suitable excipients which could be included with a carrier composition or a formulation of the present invention. The choice of other excipients will depend on the characteristics of the biologically active compound and the form of administration used. Examples of other excipients include water, thickeners or gelling agents, surfactants, buffers, emollients (organic solvents), sweeteners, disintegrators, flavours, colours, fragrances, electrolytes, pH modifiers, appearance modifiers, film foaming polymers, and the like. Suitable sweeteners include sucrose, lactose, glucose, aspartame or saccharin. Suitable disintegrators include corn starch, methylcellulose, polyvinylpyrrolidone, xanthan gum, bentonite, alginic acid or agar. Suitable flavours include peppermint oil, oil of wintergreen, cherry, orange or raspberry flavouring. The relatively high concentration of organic solvent may avoid the need for a further preservative to be added; however if considered necessary, any suitable preservatives known to a person skilled in the art may be added including but not limited to sodium benzoate, methylparaben, propylparaben, and sodium bisulphite. Excipients may be added during any step of the preparation process, usually after addition of the water.

The amount of excipient or excipients present is from 0% w/w up to about 10% w/w, up to about 5% w/w, up to about 3% w/w, or within the range of about 3% w/w to about 5% w/w, of the total concentration of the carrier composition.

### FIGURES

The examples will be described with reference to the accompanying figures in which:

FIG. 1 is a graph providing results relevant to the comparative study of Example 1;

FIG. 2 is a graph providing results relevant to Example 2;

FIG. 3 is a graph providing results relevant to Example 2; and

FIG. 4 is a graph providing results relevant to Example 3.

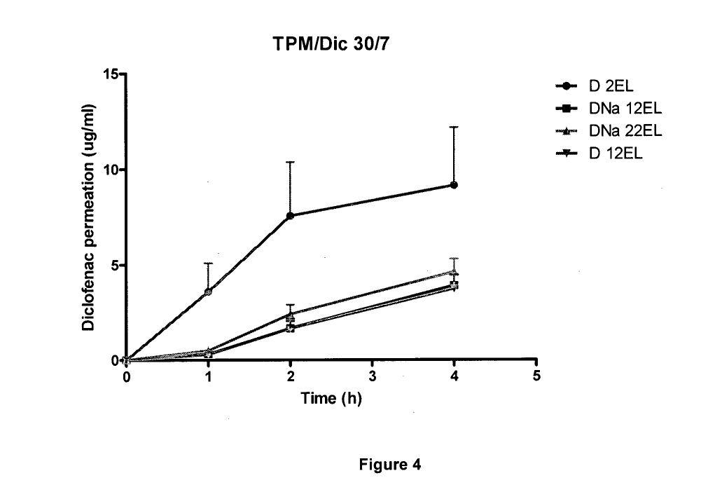

### EXAMPLES

Various embodiments/aspects of the present invention will now be described with reference to the following non-limiting examples.

### Example 1

### Investigation into the Dermal Absorption of Coenzyme-Q10 (CoQ10) Formulated with Carrier Compositions Comprising Tocopheryl Phosphate (TPM) and Various Solvents

### Method

It was determined that Nivea Visage® (control) comprises 0.05 w/w % CoQ10.

Based on this, the following three formulations, prepared according to the method described above, were tested.

### Comparative Ethanolic Formulation (pH 4.5)

| Component | Amount (w/w %) |
|---|---|
| CoQ10 | 0.05 |
| TPM | 1 |
| ethanol | 10 |
| Carbopol in water | 0.5 |

### Formulation (A) (pH 4.5)

| Component | Amount (w/w %) |
|---|---|
| CoQ10 | 0.05 |
| TPM | 1 |
| N-methyl-2-pyrrolidone (NMP) | 10 |
| Carbopol in water | 0.5 |

### Formulation (B) (pH 4.5)

| Component | Amount (w/w %) |
|---|---|
| CoQ10 | 0.05 |
| TPM | 1 |
| dimethyl isosorbide (DMI) | 10 |
| Carbopol in water | 0.5 |

### In Vitro Testing of Formulations Using Franz Diffusion Cells

Full thickness human skin was obtained from abdominoplasty procedures for use in 12 ml vertical Franz diffusion cells (Permegear, PA). All underlying fat and connective tissue was removed. Skin was frozen flat between sheets of aluminium foil and stored at −20° C. until the morning of experimentation.

Franz cells used PBS as the receptor solution (12 ml) and had a surface area of 1.77 cm². During experiments, the cells were maintained at 32° C. Finite dosing (40 μl per cell) of each formulation was used to approximate a large dose used in vivo. Twenty four cells were used, with n=4-5 cells per treatment group. Receiver solutions were sampled regularly over 4 hours to determine the percutaneous CoQ10 absorption. At the conclusion of the 4 hour period, the skin was removed from the Franz cells and the unabsorbed drug remaining on the surface carefully washed off. The absorbed CoQ10 was then extracted from the skin using 1-propanol. Time-points from the diffusion cells and skin extract were loaded onto HPLC plates for quantitation of CoQ10 content using a validated method.

TABLE I

| | | | | Formulation | Formulation |
|---|---|---|---|---|---|
| | | | Comparative | of the | of the |
| | | Nivea | ethanolic | invention | invention |
| | Untreated | Visage ® | formulation | (A) | (B) |
| Mean (±SD) | 496.65 | 546.24 | 1464.00 | 1024.80 | 1679.25 |
| | (±48.55) | (±183.07) | (±399.64) | (±76.80) | (±281.63) |
| P value vs | | | | | |
| untreated | | 0.618688 | 0.00209 | 2.44E−05 | 0.000168 |
| P value vs | | | | | |
| Nivea | | | | | |
| Visage ® | | | 0.001606 | 0.001867 | 0.000159 |
| % increase | | 9.98 | 194.77 | 106.34 | 238.12 |
| over | | | | | |
| endogenous | | | | | |
| CoQ10 | | | | | |
| % increase | | | 1950.70 | 1065.03 | 2384.75 |
| over Nivea | | | | | |
| Visage ® | | | | | |

### Results

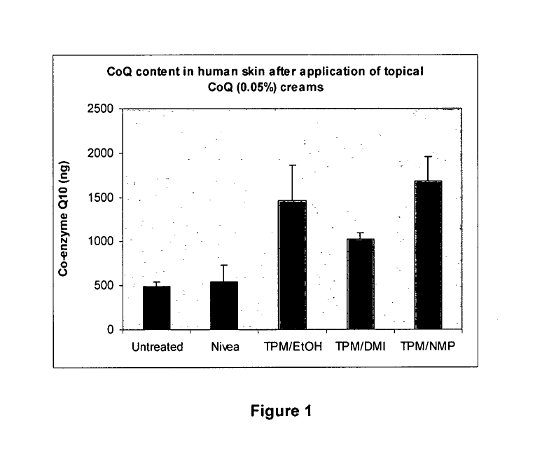

The comparative ethanolic formulation and the formulations of the present invention (A) and (B) increased the amount of CoQ10 detected in the skin compared to endogenous levels within the untreated skin samples (see FIG. 1). These increases were in the range of 106-238% (refer to Table I below) and were all statistically significant (p<0.005). In comparison, average CoQ10 levels after treatment with Nivea Visage® were increased by only ˜10%, which is not considered significant. The comparative ethanolic formulation and the formulations of the present invention (A) and (B) produced significant increases (p<0.002) in the amount of CoQ delivered to the skin over the Nivea Visage® (see Table). These increases ranged from 190-310%.

### Conclusion

This example showed that carrier compositions comprising TPM and a polar aprotic solvent provide a useful and more stable alternative to a carrier composition comprising TPM and ethanol.

### Example 2

### Investigation into the Pharmacodynamics of Insulin Formulated with TPM and Ethyl Lactate

### Method

### Negative Control—KY Jelly

Used to monitor the reduction in blood glucose caused by the physical process of rubbing a topical formulation onto a rat. Stress can cause wide fluctuations in blood glucose.

### Positive Control—TPM/Insulin Using Ethanol as Solvent

Standard TPM/insulin formulation created by Phosphagenics used in phase I and II clinical trials for efficacy. This formulation reproducibly reduces blood glucose in the STZ rat model. This formulation contained 2.26 mg/ml insulin, 30% ethanol as the solvent, 2% TPM (2:1) and 1% hydroxypropylcellulose H in water.

### TPM/Insulin EL 1-3

Three different TPM/insulin formulations were tested containing ethyl lactate as a substitute for ethanol. The formulations (all pH 7) contained:
IN#1: 12 mg/ml insulin, 4% ethyl lactate, 4% TPM (2:1) and 1% hydroxypropylcellulose H in water.
IN#2: 12 mg/ml insulin, 15% ethyl lactate, 2% TPM (2:1) and 1% hydroxypropylcellulose H in water.
IN#3: 2.26 mg/ml insulin, 4% ethyl lactate, 4% TPM (2:1) and 1% hydroxypropylcellulose H in water.

### Treatment Application

This study was designed to test the effect of a new TPM/insulin formulation containing ethyl lactate on glucose homeostasis in streptozotocin-treated diabetic rats in order to determine the optimum dose. The animals (n=15) were male and 10-12 weeks of age. All animals were >300 g in weight, and had circulating glucose concentrations of >10 mmol/L in the fasted state (mean fasted glucose concentration was 22.20±2.96 mmol/L). The key endpoint of the study was blood glucose levels during a 5-hour insulin tolerance test, conducted as described below.

### Streptozotocin Administration

Diabetes was induced by the administration of a single intraperitoneal injection of streptozotocin (STZ) 50 mg/kg (Sigma Chemicals) dissolved in sodium citrate buffer (0.1 mol/L, pH 4.5) immediately before use. Rats were considered diabetic and included in the study if their blood glucose was greater than 16 mmol/L 24 hours after the STZ injection. In all groups blood glucose measurements were made by obtaining a spot sample from tail tipping. Animals were left for 5 days following STZ administration prior to testing.

### Gel Application

24 hours before the application of the formulations the animals were anaesthetised and ˜30 cm² of fur was shaved from the back, avoiding any damage to the skin that could enhance absorption of the formulations. TPM/insulin was applied at a dose of 12 mg/cm² across the shaved area. The insulin tolerance tests were performed 24 h after removing the fur. Following each treatment, the animals were allowed to recover for 3 days before the next treatment.

### ITT (Insulin Tolerance Test)

Animals were fasted for 2 hours prior to the application of insulin or control formulations. Spot blood samples were taken from the tail at 0, 30, 60, 90, 120, 180, 240 and 300 minutes after the application of formulations. Blood glucose levels were determined at the same time points using glucose sticks (AccuChek, Roche Diagnostics).

### Results

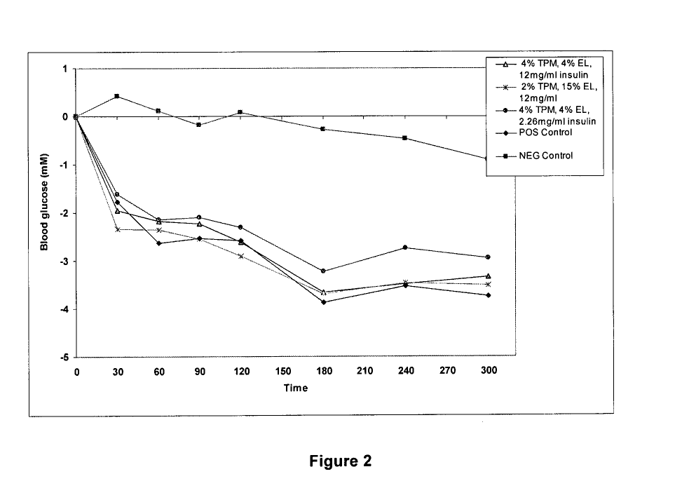

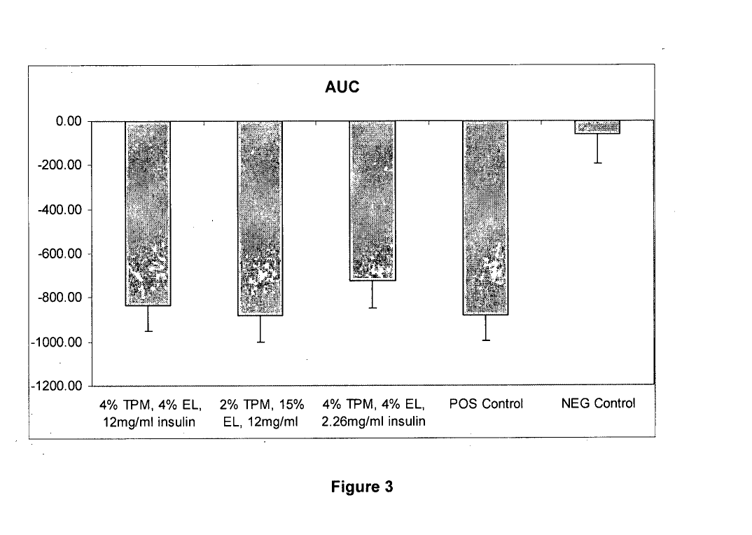

All TPM/insulin formulations caused significant reductions in blood glucose concentrations in the diabetic rats (see FIG. 2). Blood glucose was significantly reduced (p<0.05) from starting values 30 min after application and remained lowered for the duration of the experiment. There was no difference in the reduction of blood glucose between the formulations tested here, as demonstrated by the area under the curve (see FIG. 3). Ethyl lactate concentrations as low as 4%, would therefore appear as effective as ethanolic concentrations of 30%. The increased hydrophilic phase of the ethyl lactate formulation also allows increased protein concentration, although in this particular formulation the increased insulin content has not translated to increased efficacy.

### Conclusion

Ethyl lactate is able to replace ethanol in TPM/insulin formulations with no impairment in transdermal delivery. Significantly, ethyl lactate concentrations as low as 2 or 4% may be used to replace 30% ethanol, which potentially allows higher concentrations of hydrophilic drugs and a more aqueous environment to ensure the fidelity of unstable molecules such as proteins.

### Example 3

### Investigation into the Transdermal Delivery of Diclofenac Formulated with TPM and Ethyl Lactate

### Method

Four different diclofenac formulations were tested containing TP and ethyl lactate. Both the diclofenac diethylamine (D) and sodium salt (DNa) forms were used. The formulations were as follows:
DICLO#1: 5% diclofenac diethylamine, 1% TPM (6:4), 2% ethyl lactate, 1% hydroxypropylcellulose H in water.
DICLO#2: 5% diclofenac sodium salt, 1% TPM (6:4), 12% ethyl lactate, 1% hydroxypropylcellulose H in water.
DICLO#3: 5% diclofenac sodium salt, 1% TPM (6:4), 22% ethyl lactate, 1% hydroxypropylcellulose H in water.
DICLO#4: 5% diclofenac diethylamine, 1% TPM (6:4), 12% ethyl lactate, 1% hydroxypropylcellulose H in water.

### In Vitro Testing of Formulations Using Franz Diffusion Cells

Approval for animal experiments was granted by Monash University School of Biological Sciences animal ethics committee (protocols BAM/B/2006/31). Full thickness rat abdominal skin was used in 12 ml vertical Franz diffusion cells (Permegear, PA). Rats were killed by asphyxiation using CO₂ gas and the abdominal area carefully shaved and excised. All underlying fat and connective tissue was removed. Skin was frozen flat between sheets of aluminium foil and stored at −20° C. until the morning of experimentation.

Franz cells used PBS as the receptor solution (12 ml) and had a surface area of 1.77 cm². During experiments, the cells were maintained at 32° C. Finite dosing (40 μl per cell) of each formulation was used to approximate a large dose used in vivo. Twenty four cells were used, with n=4-5 cells per treatment group. Receiver solutions were sampled regularly over 4 hours to determine the percutaneous diclofenac absorption. Time-points from the diffusion cells and skin extract were loaded onto HPLC plates for quantitation of diclofenac content using a validated method.

### Results

The formulations containing ethyl lactate were able to induce transdermal diclofenac delivery. Both the diethylamine and sodium forms of the molecule were able to pass the skin. Interestingly, the formulation with the lowest ethyl lactate concentration (2%) was able to produce the best transdermal delivery.

### Example 4

### Formulation Examples

The following are three formulation examples.

### 1. Day Cream

Aqua (Purified Water)
Acetyl Octapeptide-3
Ethylhexyl Methoxycinnamate
Glycerin
C12-15 Alkyl Benzoate
Cetearyl Alcohol (and) Ceteareth-20
Benzophenone-3
Octyl Salicylate
Propylene Glycol
d-Alpha-tocopheryl phosphates (mixture of mono and di phosphates)
Glyceryl Stearate (and) PEG-100 Stearate
Dimethicone
Caprylyl Glycol
Phenoxyethanol
Tocopherol
Xanthan Gum
Hydroxyethylcellulose
Citrus Aurantium Dulcis (Orange) Oil
Carnosine
Retinol
Polysorbate 20
Camellia Sinensis (Green Tea) Extract
Santalum Album (Sandalwood) Oil
Disodium EDTA
Ubiquinone
d-Limonene
Farnesol
Linalool

### 2. Night Cream

Aqua (Purified Water)
Glycerin
C12-15 Alkyl Benzoate
Cetearyl Alcohol (and) Ceteareth-20
Caprylic/Capric Triglyceride
Acetyl Octapeptide-3
Dimethicone
d-Alpha-tocopheryl phosphates (mixture of mono and di phosphates)
Cetyl Alcohol
Caprylyl Glycol
Tocopherol
Retinol
Polysorbate 20
Carnosine
Hydroxyethylcellulose
Triethanolamine
Xanthan Gum
Magnesium Aluminum Silicate
Ubiquinone
Phenoxyethanol
Disodium EDTA

### 3. Wrinkle Freeze

Water [aqua]
Aloe barbardensis [aloe vera]
Cetearly Olivate
Glycerol Oleate
Decyl cuvate
d-Alpha-tocopheryl phosphates (mixture of mono and di phosphates)
Squalene [Olive]
Disodiumj Laurininodipropionate Tocopheryl Phosphates
Tocopherol [alpha, delta, gamma, beta natural vitamin E]
Pullulan
Oryza Sativa [Rice] Bran Oil (and) Tocotrienols
Beta carotene [Vitamin A]
Glyceryl Linoleate [Omega 6 fatty acids Vitamin F]
Aminobutyric acid
Glyceryl monosterate
Bisabolol
Reservatrol
Vitamin A palmitate
Colecalciferol [Vitamin D3]
Carageenan
Menaquinone[Vitamin K]
Ascorbyl palmitate [Vitamin C]
Lactyic Acid
Phenoxyethanol
Benzyl Alcohol
Dehydroacetic acid

In this specification, except where the context requires otherwise, the words “comprise”, “comprises”, and “comprising” mean “include”, “includes”, and “including” respectively, i.e. when the invention is described or defined as comprising specified features, various embodiments of the same invention may also include additional features.

Although this invention has been described by example and with reference to possible embodiment thereof, it is to be understood that modifications or improvements may be made thereto without departing from the scope of the invention.

1. A carrier composition for delivery of a biologically active compound comprising a phosphate compound of an electron transfer agent selected from the group consisting of mono-(tocopheryl)phosphate, mono-(tocopheryl)phosphate monosodium salt, mono-(tocopheryl)phosphate disodium salt, di-(tocopheryl)phosphate, di-(tocopheryl)phosphate monosodium salt, or a mixture thereof and a solvent selected from the group consisting of N,N-dimethyl-formamide (DMF), N-methyl-2-pyrrolidone (NMP), dimethylsulfoxide (DMSO), N,N-dimethylacetamide (DMAC), dimethyl sulfoxide, dioxane hexamethylphosphorotriamide, propylene carbonate, gamma-butyrolacetone, monomethyl ether acetate, ethyl lactate, 1,3 dimethyl-2-imidazolidinone (dimethyl isosorbide, or DMI), isopropyl myristate, propylene glycol ricinoleate, isononyl isononanoate and sucrose esters of fatty acids, isopropyl myristate, isopropyl palmitate, isopropyl isostearate, diisopropyl adipate, diisopropyl dimerate, maleated soybean oil, octyl palmitate, cetyl lactate, glycerine, caprylyl glycol, squalene, bisabolol, benzylalcohol, cetyl ricinoleate, cetyl acetate, wheat germ glycerides, myristyl lactate, decyl oleate, isopropyl lanolate, pentaerythrityl tetrastearate, neopentylglycol dicaprylate/dicaprate, isononyl isononanoate, isotridecyl isononanoate, myristyl myristate, octyl dodecanol, and octyl hydroxystearate, wherein the solvent concentration is from about 0.05% w/w up to about 30% w/w of the total concentration of the carrier composition.

2. The carrier composition of claim 1 wherein the tocopheryl phosphate is a non-neutralised form.

3. The carrier composition of claim 2 wherein the non-neutralised tocopheryl phosphate has a pH in the range of about 2 to about 4, about 2 to about 3, about 2 or about 3.

4. The carrier composition of claim 1 wherein the carrier composition comprises a mixture of a mono-(tocopheryl)phosphate to a di-(tocopheryl)phosphate in a ratio of at least 2:1, within the range of about 4:1 to about 1:4, within the range of about 6:4 to about 8:2, about 6:4, or about 8:2.

5. The carrier composition of claim 1 wherein the phosphate compound of the electron transfer agent is in an amount within the range of about 0.01% w/w to about 20% w/w, about 0.01% w/w to about 10% w/w, about 0.01% w/w to about 5% w/w, or about 0.05% w/w to about 2% w/w, of the total concentration of the carrier composition.

6. The carrier composition of claim 1 wherein the phosphate compound of the electron transfer agent is in an amount of about 0.1% w/w, about 1% w/w, or about 5% w/w, of the total concentration of the carrier composition.

7. The carrier composition of claim 1 wherein the solvent is N-methyl-2-pyrrolidone (NMP), ethyl lactate, or 1,3 dimethyl-2-imidazolidinone (dimethyl isosorbide, or DMI).

8. The carrier composition of claim 1 wherein the solvent concentration is from about 0.05% w/w up to about 20% w/w, up to about 10% w/w, up to about 5% w/w, up to about 3% w/w, up to about 2% w/w, or up to about 1% w/w.

9. Use of a phosphate compound of an electron transfer agent selected from the group consisting of mono-(tocopheryl)phosphate, mono-(tocopheryl)phosphate monosodium salt, mono-(tocopheryl)phosphate disodium salt, di-(tocopheryl)phosphate, di-(tocopheryl)phosphate monosodium salt, or a mixture thereof and a solvent selected from the group consisting of N,N-dimethyl-formamide (DMF), N-methyl-2-pyrrolidone (NMP), dimethylsulfoxide (DMSO), N,N-dimethylacetamide (DMAC), dimethyl sulfoxide, dioxane hexamethylphosphorotriamide, propylene carbonate, gamma-butyrolacetone, monomethyl ether acetate, ethyl lactate, 1,3 dimethyl-2-imidazolidinone (dimethyl isosorbide, or DMI), isopropyl myristate, propylene glycol ricinoleate, isononyl isononanoate and sucrose esters of fatty acids, isopropyl myristate, isopropyl palmitate, isopropyl isostearate, diisopropyl adipate, diisopropyl dimerate, maleated soybean oil, octyl palmitate, cetyl lactate, glycerine, caprylyl glycol, squalene, bisabolol, benzylalcohol, cetyl ricinoleate, cetyl acetate, wheat germ glycerides, myristyl lactate, decyl oleate, isopropyl lanolate, pentaerythrityl tetrastearate, neopentylglycol dicaprylate/dicaprate, isononyl isononanoate, isotridecyl isononanoate, myristyl myristate, octyl dodecanol, and octyl hydroxystearate, in the preparation of a carrier composition for delivery of a biologically active compound, wherein the solvent concentration is from about 0.05% w/w up to about 30% w/w of the total concentration of the carrier composition.

10. A process for the preparation of a carrier composition for delivery of a biologically active compound which comprises the step of combining a phosphate compound of an electron transfer agent selected from the group consisting of mono-(tocopheryl)phosphate, mono-(tocopheryl)phosphate monosodium salt, mono-(tocopheryl)phosphate disodium salt, di-(tocopheryl)phosphate, di-(tocopheryl)phosphate monosodium salt, or a mixture thereof and a solvent selected from the group consisting of N,N-dimethyl-formamide (DMF), N-methyl-2-pyrrolidone (NMP), dimethylsulfoxide (DMSO), N,N-dimethylacetamide (DMAC), dimethyl sulfoxide, dioxane hexamethylphosphorotriamide, propylene carbonate, gamma-butyrolacetone, monomethyl ether acetate, ethyl lactate, 1,3 dimethyl-2-imidazolidinone (dimethyl isosorbide, or DMI), isopropyl myristate, propylene glycol ricinoleate, isononyl isononanoate and sucrose esters of fatty acids, isopropyl myristate, isopropyl palmitate, isopropyl isostearate, diisopropyl adipate, diisopropyl dimerate, maleated soybean oil, octyl palmitate, cetyl lactate, glycerine, caprylyl glycol, squalene, bisabolol, benzylalcohol, cetyl ricinoleate, cetyl acetate, wheat germ glycerides, myristyl lactate, decyl oleate, isopropyl lanolate, pentaerythrityl tetrastearate, neopentylglycol dicaprylate/dicaprate, isononyl isononanoate, isotridecyl isononanoate, myristyl myristate, octyl dodecanol, and octyl hydroxystearate, wherein the solvent concentration is from about 0.05% w/w up to about 30% w/w of the total concentration of the carrier composition.

11. A formulation comprising a carrier composition of claim 1 and a biologically active compound.

12. The formulation of claim 11 wherein the biologically active compound is a pharmaceutical or pharmaceutically acceptable derivative thereof, a nutraceutical or nutraceutically acceptable derivative thereof, or a cosmeceutical or cosmeceutically acceptable derivatives thereof.

13. The formulation of claim 11 wherein the biologically active compound is lidocaine, diclofenac, ketoralac, prilocalne, halobetasol, hydrocortisol or combinations thereof.

14. The formulation of claim 11 wherein the biologically active compound is present in an amount of from about 0.001% w/w up to about 15% w/w, up to about 10% w/w, up to about 5% w/w, up to about 2% w/w, or up to about 1% w/w, of the total concentration of the carrier composition.

15. The formulation of claim 11 wherein the biologically active compound is present in an amount within the range of from about 0.001% w/w up to about 0.05% w/w, up to about 0.1% w/w, up to about 1% w/w, up to about 2% w/w, up to about 5% w/w, or about 1% w/w, about 2% w/w, or about 5% w/w, of the total concentration of the carrier composition.

16. A process for the preparation of a formulation of claim 11 which comprises the step of adding the biologically active compound to the carrier composition.

17. A method for treating a subject for a pathological condition, the method comprising administering an effective amount of a biologically active compound in a carrier composition of claim 1.

18. Use of a carrier composition of claim 1 to deliver a biologically active compound to treat a pathological condition in a subject.

19. Use of a carrier composition of claim 1 to improve the delivery and/or bioavailability of a biologically active compound.

20. The use of claim 19 wherein the delivery is an enteral or a parental route of administration.

21. Use of a solvent selected from the group consisting of N,N-dimethyl-formamide (DMF), N-methyl-2-pyrrolidone (NMP), dimethylsulfoxide (DMSO), N,N-dimethylacetamide (DMAC), dimethyl sulfoxide, dioxane hexamethylphosphorotriamide, propylene carbonate, gamma-butyrolacetone, monomethyl ether acetate, ethyl lactate, 1,3 dimethyl-2-imidazolidinone (dimethyl isosorbide, or DMI), isopropyl myristate, propylene glycol ricinoleate, isononyl isononanoate and sucrose esters of fatty acids, isopropyl myristate, isopropyl palmitate, isopropyl isostearate, diisopropyl adipate, diisopropyl dimerate, maleated soybean oil, octyl palmitate, cetyl lactate, glycerine, caprylyl glycol, squalene, bisabolol, benzylalcohol, cetyl ricinoleate, cetyl acetate, wheat germ glycerides, myristyl lactate, decyl oleate, isopropyl lanolate, pentaerythrityl tetrastearate, neopentylglycol dicaprylate/dicaprate, isononyl isononanoate, isotridecyl isononanoate, myristyl myristate, octyl dodecanol, and octyl hydroxystearate in an amount of 0.05% w/w up to about 30% w/w to increase the solubility and/or stability of a phosphate compound of the electron transfer agent selected from the group consisting of mono-(tocopheryl)phosphate, mono-(tocopheryl)phosphate monosodium salt, mono-(tocopheryl)phosphate disodium salt, di-(tocopheryl)phosphate, di-(tocopheryl)phosphate monosodium salt.
